(19) United States
(12) Patent Application Publication
(10) Pub. No.: US 2011/0046893 A1
(43) Pub. Date: Feb. 24, 2011

(54) METHOD FOR NORMALIZING THE RESULTS OF AN IN-VITRO ANALYTICAL METHOD

(76) Inventors: Arne HENGERER, Mohrendorf (DE); Marcus Pfister, Bubenreuth (DE); Sebastian Schmidt, Weisendorf (DE)

Correspondence Address: HARNESS, DICKEY & PIERCE, P.L.C., P.O.BOX 8910, RESTON, VA, 20195, US

(21) Appl. No.: 12/859,429

(22) Filed: Aug. 19, 2010

(30) Foreign Application Priority Data
Aug. 20, 2009 (DE) 10 2009 038 240.2

(51) Int. Cl. G06F 19/00; G01N 33/48

(52) U.S. Cl. 702/19

(57) ABSTRACT

A method is disclosed for normalizing the results of an in-vitro analytical method for one or more diagnostically and/or prognostically relevant substances in an organism (biomarker) or one or more substances supplied to an organism from the outside. In at least one embodiment of the method a) one or more concentration values of the substance(s) in an organism, said concentration value(s) being obtained in an in-vitro analytical method is (are) provided, b) data obtained from an imaging study of the same organism are provided, c) from the data according to b), one or more quantitative values are determined as imaging value(s), d) from the values according to a) and c), one or more diagnostic parameters are determined by relating the values according to a) and c) to one another, and also to the use of this method for the type-correct dosage finding of drugs and for the in-vitro diagnosis, prognosis and monitoring the course of a disease. In addition, at least one embodiment of the invention relates to a program for a data processing system which, when carried out in a suitable computer system, carries out the above method, and also to an electronically readable data carrier having electronically readable control information stored thereon, which control information is configured in such a manner that they carry out the above method when a suitable data carrier is used in a suitable computer system.

### PRIORITY STATEMENT

The present application hereby claims priority under 35 U.S.C. §119 on German patent application number DE 10 2009 038 240.2 filed Aug. 20, 2009, the entire contents of which are hereby incorporated herein by reference.

### FIELD

At least one embodiment of the invention generally relates to a method for normalizing the results of an in-vitro analytical method for substances in an organism, wherein the results are obtained from imaging methods. In particular, at least one embodiment of the invention relates to a method for normalizing the results of an in-vitro diagnostic method. In addition, at least one embodiment of the invention relates to the use of such a method for finding the type-correct dosage of drugs, and also for in-vitro diagnosis of a disease. At least one embodiment of the invention additionally relates to a program for a data processing system having a computing unit which is constructed for carrying out at least one embodiment of the method, and also to an electronically readable data carrier having electronically readable control information for carrying out at least one embodiment of the method.

### BACKGROUND

The use of metabolic biomarkers for in-vitro diagnosis of diseases is a standard method in clinical diagnosis. For instance, on the basis of, for example, the determination of biomarkers in body fluids such as blood, saliva, urine, etc., disorders or diseases of an organism may be diagnosed or prognosticated.

Markers for in-vitro diagnosis are distributed systemically in the organism. They reflect the status or sensitivity of the patient overall, or the status of an entire organ, but not always the status of a specific organ volume. This leads to unclear results if the size of an organ or function thereof is also variable under physiological conditions. This thereby leads to overlapping of the usual range of values with the pathological range of values of a marker specific for this organ.

The volume of an organ can only be determined by imaging studies. However, by way of an imaging study, only a very restricted number of somewhat unspecific biological markers can be determined, for example radiation density of magnetic properties of a tissue, whereas in-vitro analytical methods and in-vitro diagnostic methods are very specific and are available for a large number of biological markers or analytes. One example of only limited meaningfulness of the determination of concentration of an in-vitro diagnostic marker is the prostate-specific antigen (PSA) which shows high variability even with healthy people. This variability restricts the meaningfulness of a PSA determination for diagnosing cancer.

In practice, this problem is currently solved by repeating a diagnostic test after a certain period of time when the values of a marker do not lie clearly above or below an exclusion value. However, this procedure has the disadvantage that valuable time is frequently lost until a reliable diagnosis is established.

There is therefore a requirement for an improved method by which the results of an in-vitro analytical method, and in particular an in-vitro diagnostic method, can be evaluated, corrected, made more meaningful and more comparable.

### SUMMARY

in at least one embodiment of the present invention, a method is provided by which the results of an in-vitro analytical method can be made more meaningful. In particular, the method should permit the normalization of the results of an in-vitro diagnostic method and thus make the diagnostic method more meaningful. The method should be universally applicable to any desired substances which can occur in an organism. In particular, the method should be applicable to determining any desired diagnostically and/or prognostically relevant substances. The method should, in addition, be suitable for determining substances supplied from outside an organism. The method should furthermore permit improved, and in particular type-correct, finding of dosage of drugs, and should in addition permit improved and type-correct in-vitro and in-vivo diagnosis of diseases.

The method should in addition be simple to carry out, and be suitable for routine diagnosis. It should be suitable for automation.

Surprisingly, it has been found that, from the concentration values of a substance in an organism which have been obtained by means of an in-vitro analytical method, and also from data which have been obtained from an imaging method, new diagnostic parameters can be calculated which possess increased meaningfulness both with respect to the results of an in-vitro analytical method and also the results of an imaging method.

At least one embodiment of the invention therefore relates to a method for normalizing the results of an in-vitro analytical method for one or more diagnostically and/or prognostically relevant substances in an organism (biomarker) or one or more substances supplied to an organism from the outside, characterized in that a) one or more concentration values of the substance(s) in an organism, said concentration value(s) being obtained in an in-vitro analytical method is (are) provided, b) data obtained from an imaging study of the same organism are provided, c) from the data according to b), one or more quantitative values are determined as imaging value(s), d) from the values according to a) and c), one or more diagnostic parameters are determined by relating the values according to a) and c) to one another.

In addition, at least one embodiment of the invention relates to the use of this method for the type-correct dosage finding of drugs, for in-vitro diagnosis of a disease, for monitoring the course of a disease, and also for prenatal diagnosis. In addition, at least one embodiment of the invention relates to a program for a data processing system (computer program product with a computer program) which, when carried out in a suitable computer system, carries out at least one embodiment of the abovementioned method, and also to an electronically readable data carrier having electronically readable control information stored thereon, which control information are configured in such a manner that they carry out at least one embodiment of the abovementioned method when a suitable data carrier is used in a suitable computer system.

At least one embodiment of the method claimed in the invention is suitable for use for any desired organisms. Preferably, these organisms are mammals, including humans.

According to at least one embodiment of the invention, in a first step, a concentration of a substance to be determined in an organism is determined by means of an in-vitro analytical method. This substance can be a substance inherent to the body of diagnostic and/or prognostic relevance (=biomarker) or a substance supplied from the outside to the organism, such as, e.g. a drug, precursor thereof or breakdown product thereof. The concentrations of a plurality of substances can also be determined.

A biomarker is preferably a substance formed by a tissue, an organ, a gland, or a neoplasm (e.g. tumor, cancer) of the organism. These biomarkers can be detected in the tissues, organs, the gland or the neoplasm itself (for example by means of biopsy) or can be detected in body fluids such as, for example, blood, plasma, serum, urine, saliva, liquor, feces, digestive secretions, lymph, ejaculate, human milk or sputum. Examples of biomarkers of an organism are hormones, chemical messengers, tumor markers, electrolytes, vitamins, metabolic products, neurotransmitters, enzymes, etc.

Preferably, the method according to at least one embodiment of the invention is suitable for normalizing analytical methods for hormones or tumor markers. Examples of diagnostically and/or prognostically particularly relevant hormones are thyroid hormones (T3, T4), pituitary hormones (ACTH, TSH, prolactin, somatotropin), insulin or gastrin.

Examples of tumor markers are CEA (carcinoembryonic antigen), PSA (prostate-specific antigen), CA 15-3 (cancer antigen 15-3), alpha-fetoprotein.

Tests for determining such substances are known in the specialist field and are carried out in accordance with the instructions of the manufacturer.

It is also possible to determine the concentration of a substance which is supplied to an organism from the outside voluntarily or involuntarily. Examples thereof are drugs, pro-drug forms thereof or breakdown products thereof formed in the body and also poisons, virus-associated molecules or bacteria-associated molecules.

Preferably, the method according to at least one embodiment of the invention is used for normalizing the concentration values of drugs which are metabolized rapidly and have a low therapeutic width. These include, in particular, fat-soluble drugs, drugs crossing into liquor or drugs crossing the placenta of low therapeutic width. Examples thereof are psychopharmaceuticals, immunosuppressives, (local) anesthetics and β-blockers.

The drug in this case can be administered to an organism in any desired routes, e.g. orally, parenterally, topically, intravenously, intraarterially, intravascularly, intra-muscularly, etc. Examples of drugs, the distribution of which in the organism can be controlled particularly advantageously using the method according to the invention are phenytoin, cyclosporine, lidocaine, metoprolol or imipramine. Such substances can occur virtually in any region of the body. Tests for such substances are known in the specialist field and are carried out in accordance with the instructions of the manufacturer.

In a next step, the data from an imaging study of the same organism are provided. The imaging study relates here to the organ or tissue under study. Methods for carrying out an imaging method are known in the specialist field and comprise, for example, SPECT (Single Photon Emission Computed Tomography), MRI techniques, CT, PET, C-arm CT carried out using an angiography system, ultrasound or optical imaging. The method depends on the tissue, pathology and according to the value to be determined of the diagnostically/prognostically relevant substance.

From the data of an imaging study, then one or more quantitative values are determined as what are termed imaging values. Examples thereof are the (blood) volume of the organ, tissue, gland or neoplasm under study, the perfusion volume of an organ, a tissue or of a gland or a neoplasm or the body fat content of an organism. These values can be obtained using customary and suitable methods of evaluation, e.g. segmentation of data sets. These methods are well known to a person skilled in the art.

From the concentration values obtained using an analytical method and the imaging value, then one or more new diagnostic parameters are determined, wherein the values are related to one another, e.g. as a concentration value per unit volume or as a concentration change per unit time and volume. The nature of the resultant concentration value and the imaging value gives immediately the manner in which these values are to be related to one another. The resultant value is a novel diagnostic parameter, with reference to which the presence of a disease can be diagnosed or prognosticated. This diagnostic parameter is related to corresponding values which have been obtained from a healthy population. Deviations therefrom are indicative of the occurrence or possible future occurrence of a disease or of a pathological state in an organism. From what value a diagnostic parameter already indicates the occurrence or possible future occurrence of a pathological state can readily be determined by a person skilled in the art. A practical approach in this case is that the values obtained from a healthy population are taken as a 100% value and values having a deviation of 2 to 3 percent from this value are considered to be diagnostically or prognostically relevant (i.e. the 97% value based on the value of the healthy population is taken as the exclusion limit).

Quite generally, the method according to at least one the invention is suitable not only for prognosis but also for diagnosis of diseases and for monitoring the course of the progress of a disease, for monitoring a therapy carried out and also for prenatal diagnosis.

The relationship to the size of an organ permits a concentration value obtained in an in-vitro diagnostic method to be corrected with respect to organ size, since a smaller organ could produce in such a test a normal total amount of an IVD marker, but in fact already possess a pathologically high production of the marker per milliliter.

### BRIEF DESCRIPTION OF THE DRAWINGS

Further advantages, features and properties of the present invention are explained below in more detail with the aid of example embodiments and with reference to the accompanying drawings, in which:

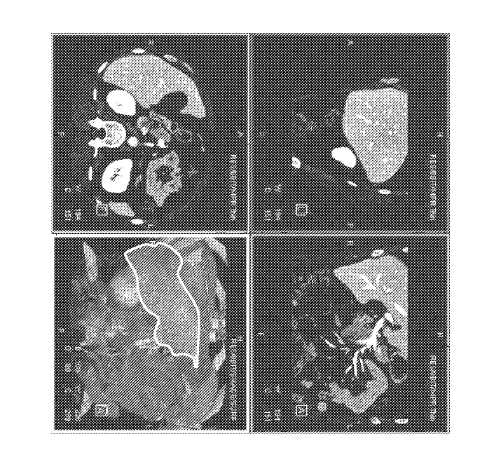

The accompanying FIG. 1 explains an embodiment of the invention in more detail.

### DETAILED DESCRIPTION OF THE EXAMPLE EMBODIMENTS

Various example embodiments will now be described more fully with reference to the accompanying drawings in which only some example embodiments are shown. Specific structural and functional details disclosed herein are merely representative for purposes of describing example embodiments. The present invention, however, may be embodied in many alternate forms and should not be construed as limited to only the example embodiments set forth herein.

Accordingly, while example embodiments of the invention are capable of various modifications and alternative forms, embodiments thereof are shown by way of example in the drawings and will herein be described in detail. It should be understood, however, that there is no intent to limit example embodiments of the present invention to the particular forms disclosed. On the contrary, example embodiments are to cover all modifications, equivalents, and alternatives falling within the scope of the invention. Like numbers refer to like elements throughout the description of the FIGURE.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of example embodiments of the present invention. As used herein, the term “and/or,” includes any and all combinations of one or more of the associated listed items.

It will be understood that when an element is referred to as being “connected,” or “coupled,” to another element, it can be directly connected or coupled to the other element or intervening elements may be present. In contrast, when an element is referred to as being “directly connected,” or “directly coupled,” to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments of the invention. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof.

It should also be noted that in some alternative implementations, the functions/acts noted may occur out of the order noted in the FIGURE. For example, two figures shown in succession may in fact be executed substantially concurrently or may sometimes be executed in the reverse order, depending upon the functionality/acts involved.

Spatially relative terms, such as “beneath”, “below”, “lower”, “above”, “upper”, and the like, may be used herein for ease of description to describe one element or feature's relationship to another element(s) or feature(s) as illustrated in the FIGURE. It will be understood that the spatially relative terms are intended to encompass different orientations of the device in use or operation in addition to the orientation depicted in the FIGURE. For example, if the device in the FIGURE is turned over, elements described as “below” or “beneath” other elements or features would then be oriented “above” the other elements or features. Thus, term such as “below” can encompass both an orientation of above and below. The device may be otherwise oriented (rotated 90 degrees or at other orientations) and the spatially relative descriptors used herein are interpreted accordingly.

Although the terms first, second, etc. may be used herein to describe various elements, components, regions, layers and/or sections, it should be understood that these elements, components, regions, layers and/or sections should not be limited by these terms. These terms are used only to distinguish one element, component, region, layer, or section from another region, layer, or section. Thus, a first element, component, region, layer, or section discussed below could be termed a second element, component, region, layer, or section without departing from the teachings of the present invention.

The accompanying FIG. 1 explains an embodiment of the invention in more detail. The sequence of pictures shows by way of example the segmentation of an organ (liver), more precisely in layers in all three spatial orientations (sagittal, coronal and transverse) and in the resultant 3D image. The organ volume is then calculated from the 3D segmentation data. The outline of the liver is marked.

In one embodiment, the imaging value is the volume of the tissue, organ, the pathology (e.g. neoplasm such as tumor), or of the gland which forms the diagnostically and/or prognostically relevant substance (diagnostic marker). The result then obtained is the amount of an in-vitro diagnostic marker per unit organ volume. Examples of combinations of imaging values and IVD values are size of the liver/liver proteins, size of the pituitary/pituitary hormones or size of the prostate/prostate-specific proteins. The organ volume can be determined, e.g. by automated segmentation or semi-automated segmentation techniques of the organ. Such methods are known in the specialist field. Segmented objects are shown, e.g., in VRT or MPR representation of the data. By means of this embodiment of the method according to the invention, diseases of the respective organ, e.g. the liver, the pituitary or the prostate, can be prognosticated or diagnosed, and also their course can be monitored.

In a further embodiment, the imaging value is the volume of the tissue, of the pathology, of the organ or of the gland on which the substance which is to be determined acts. In this embodiment, in particular subregions in the target organ are segmented and taken into account, for example what are termed “hot spots” in the pituitary, which can be identified by means of SPECT. The result obtained is then the amount of the IVD marker for hot-spot volumes. A further embodiment is determining tumor markers in relation to lesions of the breast tissue (determined by means of MR/CT), which results in an improved diagnosis and prognosis of breast cancers.

A further example in which the volume identified by the imaging study is not that of the producing organ but that of the receiving organ, is the action of insulin on fatty tissue that contains insulin receptors. A “standard” amount of insulin is insufficient to achieve “standard” reactions in obese patients. Fatty tissue can readily be identified by means of, for example, T1-weighted MR imaging on the basis of its strong signal. In addition, a number of fat-specific MRI techniques are known. The result obtained is a value “blood insulin per gram of fat”. Efficient monitoring of the course and treatment of diabetes is thereby possible.

In a further embodiment, the imaging is used to determine the distribution volume of an analyte. For example, fat-soluble drugs, such as benzodiazepines for example, accumulate in the fatty parts of the body. Therefore, in this case, the blood concentration can only be used to determine the total amount of the substance in the body if the total fat/water ratio is known. This ratio can be determined by an imaging study of a representative part of the body (for example the thorax) using a fat-selective MRI technique in which the fatty tissue is segmented and the result extrapolated to the entire body. By means of this embodiment of the invention, the distribution volume of fat-soluble substances such as drugs or environmental poisons (e.g. pesticides) in the body can be determined more accurately than is possible on the basis of an examination of blood. An improved monitoring of the course of a therapy is possible thereby.

In a further embodiment, the distribution volumes of a substance are measured in a compartment of the body by means of magnetic resonance spectroscopy (MRS) and the value compared with that value measured in the blood using an IVD test. This can be carried out repeatedly in order to determine the time point at which an equilibrium state of the distribution is achieved.

In a further embodiment of the invention, the imaging value is the perfusion volume of an organ, a tissue or a gland. Perfusion data can be obtained, for example by means of MRI. By this means the amount of an IVD marker per unit of perfused (or non-perfused) organ volume can be determined, for example the troponin release per unit of non-perfused volume of the myocardium. The perfusion can be determined either using a contrasting medium or using techniques such as ASL (Arterial Spin Labeling). The diagnosis of heart diseases (heart attack) may be improved thereby. Other functional parameters are, for example, the diffusion (by means of MRI) or the enrichment of radiotracers in nuclear medicine.

A further embodiment of the invention is the use thereof in prenatal diagnosis. By this means the concentrations of certain substances such as, for example, alpha-fetoprotein, can be determined in the blood of the mother in relation to organ volumes of the unborn child. As imaging method, in this case techniques such as ultrasound or MR are used. On the basis of the parameters thus obtained, the healthy development of an unborn child may be followed.

The method according to an embodiment of the invention can be used in order to determine the correct or type-correct dosage of drugs. For this purpose the concentration of an endogenous or exogenous test substance that demonstrates a similar behavior to the drug in question (for example hepatic elimination via the same metabolic pathway) is determined by means of an in-vitro test, whereas the imaging method is used in order to determine the distribution volume. For example, strongly lipophilic drugs accumulate in all fatty tissues including brain fat and body fat. Therefore, obese patients require an elevated dose in order to obtain an identical concentration in the brain, since their body fat mass/brain fat mass ratio is greater. However, reduced liver function could lead to a low excretion of the drug which means that the dosage must be reduced.

If the IVD marker must be determined in the organ itself (for example by means of biopsy), the result can be automatically correlated with the organ size. The morphological image (for example CT) of the patient is recorded and the recording device is connected via a known device to a 3D data set and recorded. The results can also be converted to the 3D volume and compared, for example, with the anatomical information. This would permit a detailed diagnosis of the status.

One example thereof is the determination of concentration of substances in a liver biopsy and correlation thereof to the liver volume determined by way of CT. The absolute amount of a substance in the liver may be determined thereby.

The results obtained above can also be correlated with information which is obtained from processing the 3D volume (for example automatic, manual, or semi-automatic segmentation). For example, the concentration of a certain drug or a marker may be related to the size of the organ or the region (estimated on the basis of a CT segmentation) in which it was determined.

By way of the method according to an embodiment of the invention, the results of an in-vitro analytical method, and in particular of an in-vitro diagnostic method can be made more meaningful, since the diagnostically unclear range of values of a marker is reduced. In addition, type-correct dosages of drugs can be found, and a patient-specific therapy can be developed. The method according to an embodiment of the invention therefore permits a marked improvement of diagnosis and therapy of numerous diseases.

The method according to an embodiment of the invention can be automated and carried out, e.g., using a computer program on a corresponding computing unit. A corresponding computer program can readily be developed by a person skilled in the art for the method according to an embodiment of the invention on the basis of the above disclosure.

The patent claims filed with the application are formulation proposals without prejudice for obtaining more extensive patent protection. The applicant reserves the right to claim even further combinations of features previously disclosed only in the description and/or drawings.

The example embodiment or each example embodiment should not be understood as a restriction of the invention. Rather, numerous variations and modifications are possible in the context of the present disclosure, in particular those variants and combinations which can be inferred by the person skilled in the art with regard to achieving the object for example by combination or modification of individual features or elements or method steps that are described in connection with the general or specific part of the description and are contained in the claims and/or the drawings, and, by way of combineable features, lead to a new subject matter or to new method steps or sequences of method steps, including insofar as they concern production, testing and operating methods.

References back that are used in dependent claims indicate the further embodiment of the subject matter of the main claim by way of the features of the respective dependent claim; they should not be understood as dispensing with obtaining independent protection of the subject matter for the combinations of features in the referred-back dependent claims. Furthermore, with regard to interpreting the claims, where a feature is concretized in more specific detail in a subordinate claim, it should be assumed that such a restriction is not present in the respective preceding claims.

Since the subject matter of the dependent claims in relation to the prior art on the priority date may form separate and independent inventions, the applicant reserves the right to make them the subject matter of independent claims or divisional declarations. They may furthermore also contain independent inventions which have a configuration that is independent of the subject matters of the preceding dependent claims.

Further, elements and/or features of different example embodiments may be combined with each other and/or substituted for each other within the scope of this disclosure and appended claims.

Still further, any one of the above-described and other example features of the present invention may be embodied in the form of an apparatus, method, system, computer program, computer readable medium and computer program product. For example, of the aforementioned methods may be embodied in the form of a system or device, including, but not limited to, any of the structure for performing the methodology illustrated in the drawings.

Even further, any of the aforementioned methods may be embodied in the form of a program. The program may be stored on a computer readable medium and is adapted to perform any one of the aforementioned methods when run on a computer device (a device including a processor). Thus, the storage medium or computer readable medium, is adapted to store information and is adapted to interact with a data processing facility or computer device to execute the program of any of the above mentioned embodiments and/or to perform the method of any of the above mentioned embodiments.

The computer readable medium or storage medium may be a built-in medium installed inside a computer device main body or a removable medium arranged so that it can be separated from the computer device main body. Examples of the built-in medium include, but are not limited to, rewriteable non-volatile memories, such as ROMs and flash memories, and hard disks. Examples of the removable medium include, but are not limited to, optical storage media such as CD-ROMs and DVDs; magneto-optical storage media, such as MOs; magnetism storage media, including but not limited to floppy disks (trademark), cassette tapes, and removable hard disks; media with a built-in rewriteable non-volatile memory, including but not limited to memory cards; and media with a built-in ROM, including but not limited to ROM cassettes; etc. Furthermore, various information regarding stored images, for example, property information, may be stored in any other form, or it may be provided in other ways.

Example embodiments being thus described, it will be obvious that the same may be varied in many ways. Such variations are not to be regarded as a departure from the spirit and scope of the present invention, and all such modifications as would be obvious to one skilled in the art are intended to be included within the scope of the following claims.

What is claimed is:

1. A method for normalizing the results of an in-vitro analytical method for at least one of one or more diagnostically and prognostically relevant substances in an organism (biomarker) or one or more substances supplied to an organism from the outside, that the method comprising:
a) providing one or more concentration values of the at least one substance in an organism, the one or more concentration values being obtained in an in-vitro analytical method;
b) providing data obtained from an imaging study of the same organism;
c) determining, from the data provided according to b), one or more quantitative values as one or more imaging values; and
d) determining, from the values provided according to a) and determined according to c), one or more diagnostic parameters by relating the values according to a) and c) to one another.

2. The method as claimed in claim 1, wherein the substance is a substance formed by a tissue, an organ, a gland, or a neoplasm of the organism.

3. The method as claimed in claim 1, wherein the substance is a substance acting on a tissue, an organ, a gland or a neoplasm of the organism.

4. The method as claimed in claim 1, wherein the substance is a hormone, a chemical messenger, a tumor marker, a metabolic product, a neurotransmitter or an enzyme.

5. The method as claimed claim 1, wherein the substance is a drug, pro-drug thereof, or breakdown product thereof formed in the organism.

6. The method as claimed in claim 1, wherein the imaging value is the volume or perfusion volume of a tissue, an organ, a gland or a neoplasm.

7. The method as claimed in claim 1, wherein the imaging value is the body fat content of an organism.

8. The method as claimed in claim 1, wherein the imaging method is selected from SPECT, CT, MRI techniques, PET, ultrasound, methods for optical imaging, or C-arm CT carried out using an angiography system.

9. The method as claimed in claim 1, wherein the in-vitro analytical method is an in-vitro diagnostic method.

10. A method, comprising:
using the method as claimed in claim 1 for the in-vitro diagnosis or prognosis of a disease.

11. A method, comprising:
using the method as claimed in claim 1 for prenatal diagnosis.

12. A method, comprising:
using the method as claimed in claim 1 for the type-correct dosage finding of drugs.

13. A method, comprising:
using the method as claimed in claim 1 for monitoring the course of a disease.

14. A program for a data processing system which, when carried out in a suitable computer system, carries out the method as claimed in claim 1.

15. An electronically readable data carrier including electronically readable control information stored thereon, the control information being configured to carry out the method as claimed in claim 1 when the data carrier is used in a suitable computer system.

16. The method as claimed in claim 2, wherein the substance is a hormone, a chemical messenger, a tumor marker, a metabolic product, a neurotransmitter or an enzyme.

17. The method as claimed in claim 3, wherein the substance is a hormone, a chemical messenger, a tumor marker, a metabolic product, a neurotransmitter or an enzyme.

18. The method as claimed claim 2, wherein the substance is a drug, pro-drug thereof, or breakdown product thereof formed in the organism.

19. The method as claimed claim 3, wherein the substance is a drug, pro-drug thereof, or breakdown product thereof formed in the organism.

20. A computer readable medium including program segments for, when executed on a computer device, causing the computer device to implement the method of claim 1.
